(19) United States
(12) Patent Application Publication
(10) Pub. No.: US 2025/0090368 A1
(43) Pub. Date: Mar. 20, 2025

(54) THERMALLY ACTIVATED PESSARY

(71) Applicant: Beth Israel Deaconess Medical Center, Inc., Boston, MA (US)

(72) Inventors: Mallika Anand, Brookline, MA (US); Ara Nazarian, Wellesley, MA (US)

(21) Appl. No.: 18/726,658

(22) PCT Filed: Aug. 24, 2022
(86) PCT No.: PCT/US2022/041369

§ 371 Date: Jul. 3, 2024

Related U.S. Application Data
(63) Continuation of application No. 17/445,778, filed on Aug. 24, 2021.
(60) Provisional application No. 63/069,490, filed on Aug. 24, 2020.

(51) Int. Cl. A61F 6/12; A61F 2/00

(52) U.S. Cl. CPC A61F 6/12; A61F 2/0009; A61F 2210/0028; A61F 2210/0033; A61F 2230/0091

(57) ABSTRACT

A system, method, and apparatus to insert and remove a thermally activated vaginal pessary (102), by employing a nitinol core material that is integrated into the construction of the thermally activated vaginal pessary and configured to increase in radial size upon contact with body temperature by undergoing a configurational phase change in the underlying nitinol core material.

### CROSS-REFERENCE TO RELATED APPLICATIONS

This application is a continuation-in-part of U.S. application Ser. No. 17/445,778, filed Aug. 24, 2021, which claims priority to U.S. Provisional Application No. 63/069,490, filed Aug. 24, 2020, the entire contents of the above-identified applications being incorporated herein by reference.

### TECHNICAL FIELD

The present application relates generally to a vaginal pessary for managing pelvic organ prolapse (POP) and stress urinary incontinence (SUI), and more particularly relates to a vaginal pessary that may be easily inserted and removed by a patient as needed, thereby facilitating improved hygiene, comfort, and thus reducing common pessary related issues and complications.

### BACKGROUND

The pelvic floor is a group of muscles, ligaments and connective tissues that together support the pelvic organs. In women, the pelvic floor muscles and their surrounding fasciae form a support system to keep the bladder, the rectum and the uterus in place. These muscles and fasciae are attached to the fixed framework of the pelvic bones.

Pelvic organ prolapse (POP) and stress urinary incontinence (SUI) occur when the pelvic floor muscles are weakened thereby allowing one or more of the pelvic organs to push against the weakened wall of the vagina. Although not life-threatening, POP and SUI may have significant effects related to urinary, sexual, and colorectal functions, adversely affecting quality of life. For women with significant pelvic floor symptoms, the management of POP and/or SUI consists of surgical reconstruction or vaginal pessaries.

Vaginal pessaries (i.e., plastic or silicone devices that are inserted into the vagina to help support the vaginal walls and pelvic organs) are used for POP and/or SUI reduction in women who prefer conservative, non-surgical treatment. Periodic removal of these devices, however, is necessary to maintain good hygiene and to reduce or eliminate complications caused at least in part by the prolonged presence of these devices in the vagina. Such complications including irritation, erosion, bleeding and malodorous discharge. Ideally, pessaries should be removed on a daily basis.

An issue with existing pessary designs is that removal is often uncomfortable and painful and requires the pessary to be manually squeezed or bent in order to be inserted or removed from the body, thus making self-insertion and removal difficult if not impossible, particularly for elderly patients. As a result, most patients are required to rely on lifelong regular office visits every month for removal, inspection and cleaning, and reinsertion, often resulting in painful abrasions of the vaginal introitus.

It is the object of the present invention to provide a vaginal pessary that avoids the drawbacks associated with these prior art designs.

### BRIEF DESCRIPTION OF THE DRAWINGS

Some embodiments are illustrated by way of example and not limitation in the figures of the accompanying drawings in which:

FIG. 1A is a diagram illustrating a thermally activated vaginal pessary in position upon a surface of a patient's vaginal wall, according to certain example embodiments.

FIG. 1B illustrates an alternative inner core structure for the vaginal pessary according to some embodiments taught herein.

FIG. 1C illustrates an alternative inner core structure for the vaginal pessary according to some embodiments taught herein.

FIG. 1D illustrates an alternative inner core structure for the vaginal pessary according to some embodiments taught herein.

FIG. 2A is a diagram illustrating an applicator for a thermally activated vaginal pessary in a first phase configuration, according to certain example embodiments.

FIG. 2B is a diagram illustrating an applicator for a thermally activated vaginal pessary in a second phase configuration, according to certain example embodiments.

FIG. 3A is a diagram illustrating an applicator for a thermally activated vaginal pessary, according to certain example embodiments.

FIG. 3B illustrates a handle for use with the applicator of FIG. 3A according to some embodiments taught herein.

FIG. 4 is a flowchart depicting a method of inserting a thermally activated vaginal pessary, according to certain example embodiments.

FIG. 5A illustrates a vaginal pessary including sensors in accordance with some embodiments taught herein.

FIG. 5B illustrates a ring-shaped vaginal pessary in accordance with some embodiments taught herein.

FIG. 6 illustrates a graph showing elasticity of different phases of nitinol as a function of annealing temperature in accordance with some embodiments described herein.

FIG. 7 illustrates a process flow diagram for use of a pessary device described herein to control a nerve stimulation signal and/or to use sensors to measure a characteristic of the device and/or the device environment.

### DETAILED DESCRIPTION

Example methods and systems for a thermally activated pessary and insertion device. In the following description, for purposes of explanation, numerous specific details are set forth in order to provide a thorough understanding of example embodiments. It will be evident, however, to one skilled in the art that the present invention may be practiced without these specific details. The description is not to be taken in a limiting sense but is made merely for the purpose of illustrating the general principles of the invention, since the scope of the invention is best defined by the appended claims.

A thermally activated pessary device can utilize a shape memory material that moves between two states over a temperature range in which the device, when positioned within a patient's body, moves from a first shape to a second shape as the device undergoes a temperature change to conform to the body temperature of a subject. Different materials exhibit the required differential movement to undergo the change in configuration as a function of the adjustable temperature range wherein the device transitions to the body temperature upon placement within the body. Shape memory metal alloys can comprise materials suitable for fabrication of exemplary devices as described herein. Shape memory polymer actuators can also be used to fabricate thermally activated pessary devices as described herein. In exemplary devices, the shape memory material can comprise a shaped frame within a flexible biocompatible membrane. The biocompatible membrane can comprise polymer materials or silicone materials, for example, that can serve as the tissue contact surfaces of the device. In some embodiments, the outer surface can include silver embedded into the material or coated on the material.

Embodiments of the present invention provide a system, method, and apparatus to provide a thermally activated vaginal pessary that employs a nitinol core material that is integrated into the construction of the thermally activated vaginal pessary and configured to increase in radial size upon contact with body temperature by undergoing a configurational phase change in the underlying nitinol core material. In some example embodiments, the thermally activated vaginal pessary may comprise one or more integrated sensor devices configured to communicate with a client device, including but not limited to: one or more pH analysis sensors; one or more pressure sensors, which may include a piezoresistive strain gauge, or capacitive pressure sensor; one or more volume sensors; one or more microbial sensors; a drug elution dispenser; and one or more sensors to provide sacral spinal nerve root 3 washout signal. Accordingly, portions of one or more integrated sensor devices may be embedded within a vaginal pessary, including but not limited to the thermally activated vaginal pessary.

The thermally activated vaginal pessary comprises a flexible outer surface composed of a biocompatible material, such as silicone or another polymer, and an inner core of a shape-memory metal alloy (Nitinol), wherein contact with internal body temperature at the time of insertion causes the inner core to undergo a configurational phase change from its martensitic phase to its austenitic phase, resulting in a physical change in shape that causes the thermally activated vaginal pessary to expand (increase in radial size) and press against the surfaces of the vaginal wall, thereby providing support.

In certain embodiments, the thermally activated vaginal pessary may include an applicator device configured to cool the nitinol core material of the thermally activated vaginal pessary, causing the thermally activated vaginal pessary to shrink in radial size for the purposes of easier insertion or retraction. For example, the applicator device may comprise a cooling wand that comprises a manually operated clamp configured to squeeze the thermally activated vaginal pessary while cooling the nitinol core material in order to shrink the radial size of the inner core, for removal of the pessary from the patient's vaginal cavity.

In some embodiments, the nitinol core material of the thermally activated vaginal pessary may comprise a series of nitinol ribs to be aligned in series, hemi-cylindrically, or in some embodiments may comprise a continuous spiral of nitinol helical elements along a long axis. In certain embodiments, the nitinol ribs may be connected to one another by a non-phase changing element keeping the inter-element distance constant. Accordingly, the individual components of the nitinol core may themselves be treated using a specific method based on a desired transformation result.

The treatment method may comprise a treatment temperature and a treatment duration. Accordingly, different nitinol properties may be achieved by applying different treatment processes. For example, in some embodiments, the nitinol components may be treated at a treatment temperature of 805 Celsius (C), and a treatment duration of 5 minutes. In further embodiments, the nitinol components may be treated at a treatment temperature of 550° C., and a treatment duration of 5 minutes. In further embodiments, the nitinol components may be treated at a treatment temperature of 400° C. and a treatment time of 5 minutes. In further embodiments, the nitinol components may be treated at a treatment temperature of 805° C., and a treatment duration of 30 minutes.

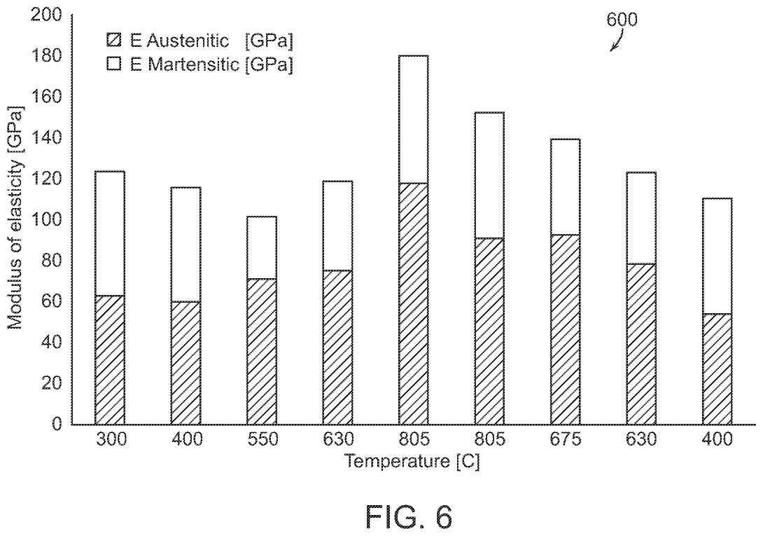

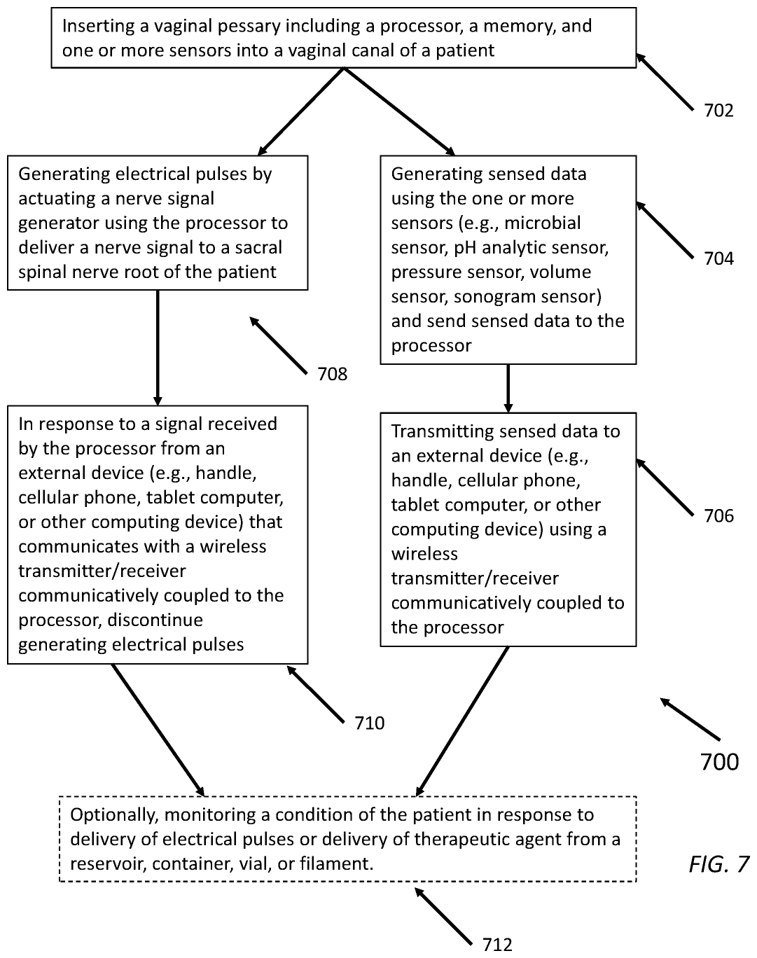

FIG. 6 is a graph 600 illustrating elasticity of different phases of nitinol as a function of annealing temperature. As shown in the graph, different annealing temperatures can affect a change in the modulus of elasticity for each state while also having an impact on the change in modulus of elasticity between the two states (i.e., austenitic and martensitic). Time spent at each temperature also plays a role in the final elasticity. In this graph 600, samples that have the same temperature but that show different modulus of elasticity characteristics were annealed for different times. Preferred methods for making embodiments of the devices shown and described herein can employ different annealing temperatures to provide different expansion ratios for the changes in temperature needed to contract and expand the structures to engage surrounding tissue upon deployment to stabilize the position of the device within the body.

In some embodiments, the treatment processes described above may also include one or more quench cycles, wherein the nitinol components are rapidly cooled in water, oil, or air. In some embodiments, the treatment process may comprise a combination of multiple treatment process cycles. For example, as an illustrative example, the treatment process for a particular nitinol component may comprise an initial treatment temperature of 805° C. for a treatment duration of 30 minutes, followed by a first quench, followed by a subsequent treatment temperature of 550° C. at a treatment duration of 5 minutes and a second quench.

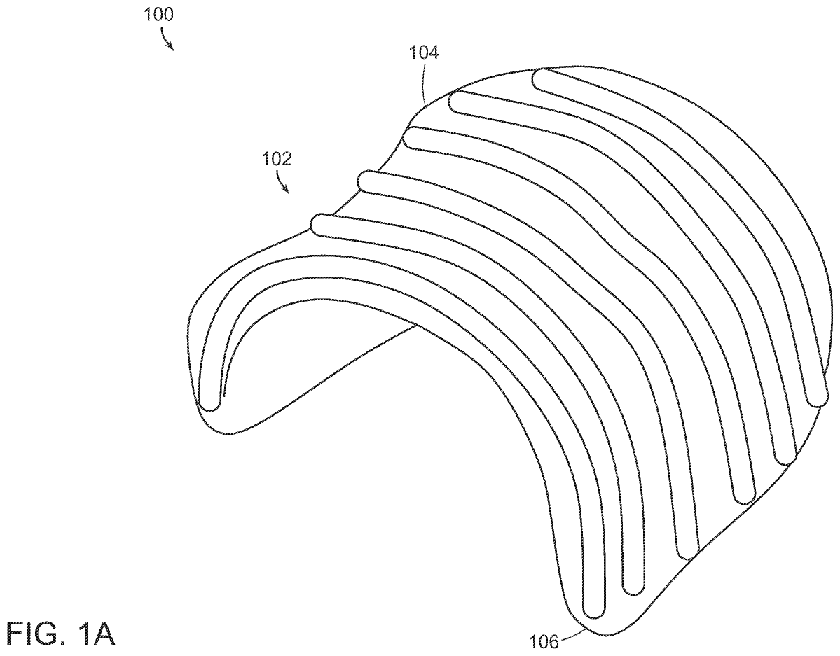

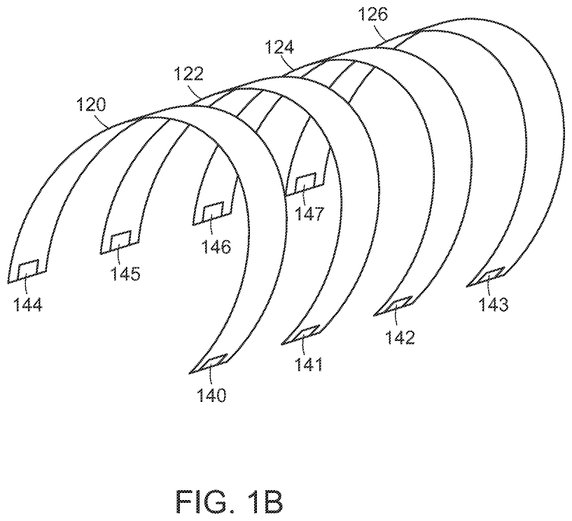

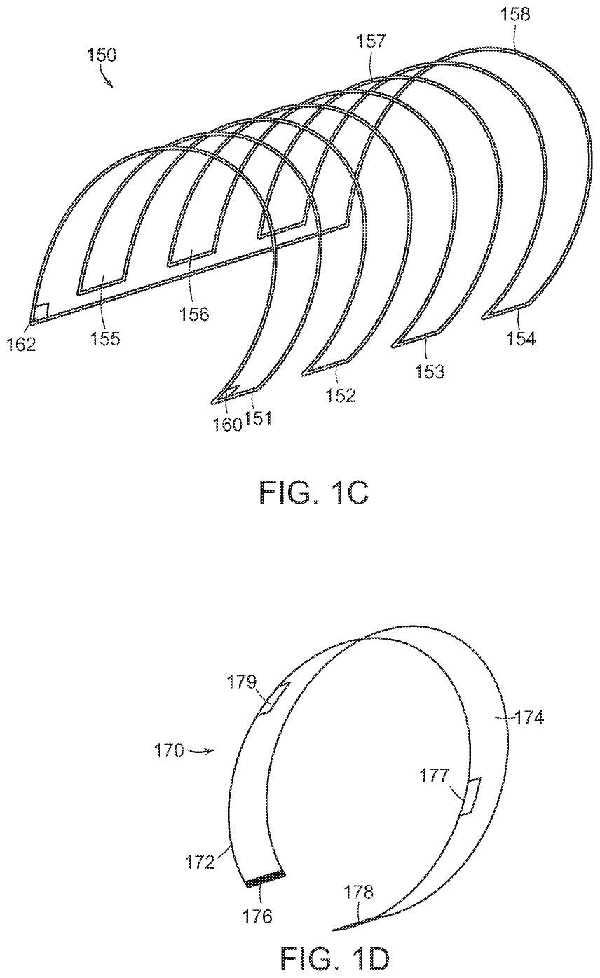

For purposes of non-limiting illustration, the drawings of FIGS. 1A-1D, 2A-2B, and 3A-3B generally depict certain example embodiments of a thermally activated vaginal pessary and associated devices.

FIG. 1A provides a diagram 100 of a thermally activated vaginal pessary 102 that comprises an outer surface 104, and a set of nitinol core elements (i.e., ribs) 106, which may be enclosed within the outer surface 104, according to certain example embodiments. In some embodiments, the set of nitinol ribs 106 may be arranged in parallel, hemi-cylindrically. In some embodiments, the set of nitinol ribs 106 may be a continuous spiral of nitinol helical elements along a long axis of the thermally activated vaginal pessary 102. In accordance with embodiments described herein, the biocompatible material forming the outer surface can be constructed of a material that can be cleaned using soap and water upon removal of the pessary 102. The lateral sides of the pessary 102

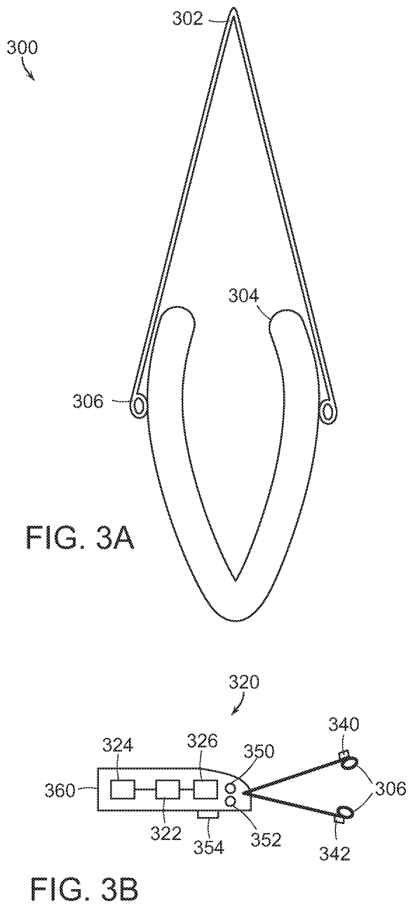

In FIGS. 1B-1D, inner core structures are depicted that are alternatives, or may be used in addition to, the nitinol core elements 106 as illustrated in FIG. 1. While the core structures of FIGS. 1B-1D are illustrated without a surrounding outer surface (e.g., made of biocompatible material), it will be understood by the person of ordinary skill in the art that the depicted inner core structures taught herein could be substituted for the ribs 106 inside the outer surface 106 to form the vaginal pessary 102. FIG. 1B illustrates an inner core structure including separate frames 120, 122, 124, 126. Each frame 120, 122, 124, 126 is formed of wires or tubes and is open in the center. The wires or tubes can be formed of the shape-memory alloy as described above. As shown in FIG. 1B, each frame can form an arch, hemi-cylindrical, or horseshoe shape. Each frame can include one or more contact pads where an external device (such a portion of an applicator, described further below with respect to FIGS. 3A and 3B) can be thermally or electrically contacted or coupled to the frame to induce temperature changes in the frame. Frame 120 includes contact pads 140, 144. Frame 122 includes contact pads 141, 145. Frame 124 includes contact pads 142, 146. Frame 126 includes contact pads 143, 147.

FIG. 1C illustrates an inner core structure formed of a single wire 150 or tube in accordance with various embodiments taught herein. The single wire 150 structure includes contact pads 160, 162 at one end to enable thermal or electrical contact or coupling with an external device such as the applicator described below with respect to FIGS. 3A and 3B to create temperature changes the wire 150. A first end of the wire 150 structure can contact a second end of the wire to create a closed figure. For example, the wire structure 150, beginning from contact pad 162, the wire can be formed into a series of arches 155, 156, 157, 158 that are connected end-to-end by longitudinal sections 151, 152, 153, 154. The final arch 158 in sequence is connected back to contact pad 162 by a length of wire or tube that returns along a longitudinal axis of the pessary. Thus, the single wire 150 can be said to form a continuous spiral of helical elements in some embodiments.

FIG. 1D illustrates a single wire frame 170 in accordance with some embodiments taught herein. The single wire frame 170 includes lower cross beams 176, 178 that approach closely to one another to approximate a single loop. The single wire frame 170 includes contact pads 174, 177 to enable thermal or electrical contact or coupling as described above. The lower cross beams 176, 178 are connected by arched wire segments 172, 174.

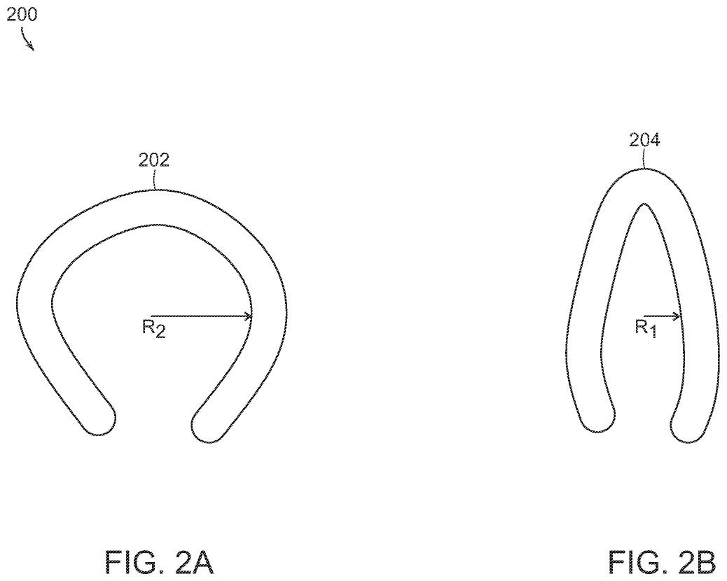

FIG. 2A provides a diagram 200 of a first phase of a thermally activated vaginal pessary, according to certain example embodiments. As depicted in the diagram 200, a first phase 202 of a thermally activated vaginal pessary (such as the thermally activated vaginal pessary 102 depicted in FIGS. 1A-1D). For example, the first phase 202 may correspond with an austenitic phase of the thermally activated vaginal pessary 102. FIG. 2B illustrates a second phase 204 of the pessary. In some embodiments, the second phase 204 of the thermally activated vaginal pessary may occur when the thermally activated vaginal pessary is cooled (i.e., a temperature of the thermally activated vaginal pessary is reduced). Accordingly, the second phase 204 may correspond with a martensitic phase of the thermally activated vaginal pessary 102.

According to certain example embodiments, the thermally activated vaginal pessary may transition from the first state 202 to the second state 204 by reducing a temperature of a nitinol ribs disposed within the thermally activated vaginal pessary (i.e., the nitinol ribs 106). The pessary 102 can be shaped and sized for insertion into the vaginal canal. When the pessary 102 warms to body temperature upon insertion, the core elements change to the configuration as shown in FIG. 2A (i.e., shape converts from the second state 204 to the first state 202) and the pessary 102 is thereby immobilized relative to the vaginal wall.

FIG. 3A provides a diagram 300 illustrating arms 302 of an applicator for a thermally activated vaginal pessary, according to certain example embodiments. As seen in the diagram 300, the arms 302 of an applicator may be configured to compress a thermally activated vaginal pessary 304 for insertion into a patient.

According to certain example embodiments, the arms 302 of the applicator device may include one or more cooling elements 306 configured to contact with an inner core of the thermally activated vaginal pessary 304, causing a phase change of the thermally activated vaginal pessary 304, as depicted in FIGS. 2A-2B. For example, the applicator device may cool the nitinol core material of the thermally activated vaginal pessary 304, causing the thermally activated vaginal pessary 304 to shrink in radial size for the purposes of easier insertion or retraction. For example, the applicator device may comprise a cooling wand that comprises a manually operated clamp (that comprises the arms 302) configured to squeeze the thermally activated vaginal pessary while cooling the thermally activated vaginal pessary 304. For example, the cooling elements 306 may comprise thermoelectric coolers. The applicator device can enable self-administration of the vaginal pessary by the patient or can assist a physician in placing the pessary.

In some embodiments, the cooling elements 306 are configured to contact or couple to the contact pads (e.g., contact pads 140, 141, 142, 143, 144, 145, 146, 147, 160, 162, 174, 177) on inner core frames or ribs as described above with respect to FIGS. 1A-1D. In some embodiments, the outer surface 104 can include apertures to enable direct contact between the cooling elements 306 and the contact pads. The apertures can be always open or can be covered by a moveable flap to protect the inner core while the pessary is in place in a patient.

FIG. 3B illustrates a handle 320 to manipulate the arms 302 of the applicator and enable ease of handling for the user. The handle 320 can enable self-administration of the pessary by a patient or can assist the physician in placing the pessary. The handle 320 can have a trigger 354 to manually adjust the spacing of the two arms 302. The arms 302 include cooling elements in the form of thermoelectric coolers (TECs) 306 near a distal end of the arms 302. A switch 326 provided on a body of the handle 320 enables actuation of the TECs 306. The handle 320 can include a battery 324 that powers a circuit board 322 within the handle 320. The circuit board 332 can control power to the TECs 306. In some embodiments, the handle 320 can include a power indicator 350 (e.g., an indicator light or display) that provides the user with the power status of the TECs 306. In some embodiments, the handle 320 can include a temperature indicator 352 (e.g., an indicator light or display) that is connected to thermocouples 340, 342 located near the distal end of the arms 302. The temperature indicator 352 can indicate when the pessary 102 has cooled sufficiently that insertion or withdrawal of the pessary from the patient can be performed. In some embodiments, the handle 352 includes a cable 360 to power the device or to charge the battery 324. In some embodiments, the battery 324 can be charged using wireless charging technology such as those compatible with the Qi® wireless charging standard.

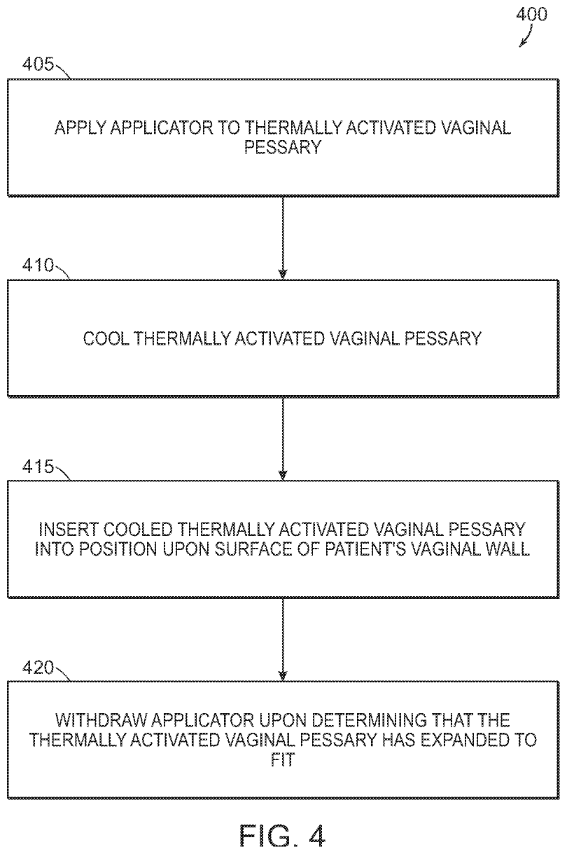

FIG. 4 depicts a method 400 of inserting a thermally activated vaginal pessary, according to certain example embodiments. As shown in FIG. 4, the method 400 may comprise operations 405, 410, 415, and 420.

At operation 405, the applicator is applied to the thermally activated vaginal pessary. According to certain embodiments discussed herein, the applicator may comprise one or more cooling elements, such as a thermoelectric cooler.

At operation 410, a temperature of the thermally activated vaginal pessary is reduced by the one or more cooling elements of the applicator, causing a nitinol core of the thermally activated vaginal pessary to change from a first phase to a second phase, wherein the second phase of the nitinol core elements corresponds with a reduction in radial size as compared with the first phase.

At operation 415, the thermally activated vaginal pessary is inserted into position upon a surface of a patient's vaginal wall. Upon determining that the thermally activated vaginal pessary has been inserted into an appropriate position as determined by an attending nurse of physician, the cooling elements of the applicator device may be disabled in order to allow the patient's body temperature to warm the thermally activated vaginal pessary.

At operation 420, the applicator may be withdrawn upon determining that the thermally activated vaginal pessary has expanded into position. In some embodiments, the applicator may include a thermocouple configured to determine when the inner core elements of the thermally activated vaginal pessary have reached a threshold temperature. For example, upon determining that the threshold temperature has been reached, a notification element associated with the applicator may display a notification indicating that the applicator may be removed. In some embodiments, the notification element may include a Light Emitting Diode (LED), or a speaker configured to emit a predefined tone.

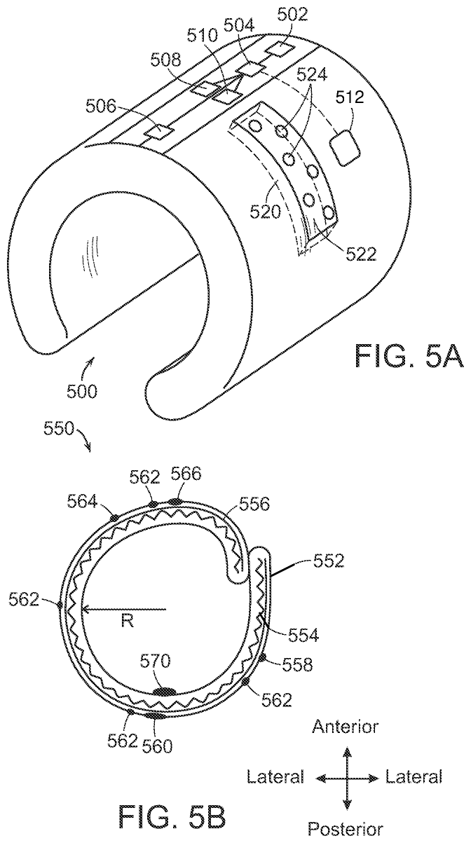

FIG. 5A illustrates a vaginal pessary 500 including one or more sensors in accordance with various embodiments taught herein. The vaginal pessary 500 can include a battery 502, a processor 504, a wireless transmitter 506, a first sensor 508, a second sensor 510, and a pressure sensor 512. The pessary 500 can use the wireless transmitter 506 to transmit data measured or sensed by the sensors 508, 510, 512 to a computing device external to the patient. The vaginal pessary 500 can also include one or more reservoirs 520 extending below a top surface of the pessary 500 that may be covered with a porous or permeable membrane 522. The membrane 522 can include one or more pores 524. The reservoir 520 can be loaded with a fluid vehicle or filament containing a medicament or other fluid that can be slowly dispensed through the membrane 522 over time. In some embodiments, the reservoir 520 can be loaded with a fluid vehicle containing estradiol such as a cream or carrier fluid that includes estradiol. In some embodiments, the reservoir 520 can be loaded with a fluid vehicle containing hyaluronic acid (HLA). In some embodiments, the reservoir 520 can be loaded with a fluid vehicle containing trimethoprim. In some embodiments, the reservoir 520 can be loaded with a fluid vehicle containing a contraceptive drug such as pharmaceutically effective doses of estrogen or progesterone. In some embodiments, the reservoir 520 can be loaded with a fluid vehicle containing a medicament to treat vaginitis which could include antibiotics in some embodiments. In some embodiments, the reservoir 520 can be loaded with a vehicle containing silver.

In some embodiments, the reservoir 520 is positioned between ribs 106 or between elements of the frames as described in FIGS. 1A-1D. In some embodiments, the reservoir 520 can hold a fluid, liquid, or semi-liquid (e.g., cream) substance. In other embodiments, the reservoir 520 can be shaped as a thin channel to receive a drug dispenser such as a solid filament impregnated with the medicament. The drug dispenser can be inserted into the channel reservoir at one end of the channel. In some embodiments, a container or vial having a cavity can be inserted into the reservoir. The cavity of the container can include a therapeutic agent to be eluted or dispensed into the body. In some embodiments, the reservoir 520, container, vial, and/or filament can be formed of a biodegradable substance that dissolves when placed within the patient's body.

The processor 504 receives data from the first sensor 508, the second sensor 510, and the pressure sensor 512. In some embodiments, data sensed by the sensors 508, 510 can include measurements of metabolic parameters such as temperature, pH, or blood flow. In other embodiments, the data sensed by the sensors 508, 510 can include secondary parameters that are indicative of patient health such as tissue color, organ volume data, pressure, microbial presence, or nerve signals. Tissue color can be assessed by a sensor 508, 510 that employs an illumination element (e.g., a light emitting diode (LED)) and an optical detector. In some embodiments, the sensor 508, 510 can sense chemical changes or other parameters as described below with respect to FIG. 5B. The pressure sensor 512 can sense a pressure between the pessary and a surface of vaginal tissue contacting the pessary. The pressure information can indicate whether the pessary 500 is properly installed and can avoid any risks associated with overpressure (which could create discomfort over time) or under pressure (which could indicate that the pessary is loose and is not sufficiently treating the patient's condition such as prolapse).

In some embodiments, one or more of the sensors 508, 510 can measure or sense properties of the pessary 500 or detect a condition of the pessary 500. For example, the first sensor 508 can measure a temperature of the inner frame (such as the ribs 106) in some embodiments to ensure that the shape-memory alloy is within the expected temperature range for the intended first state or second state shape. In some embodiments, temperature data from the inner frame is transmitted by the processor with an embedded memory 504 via the wireless transmitter/receiver 506 to the handle 320 as shown in FIG. 3B. The handle 320 can use the data received from the wireless transmitter 506 to determine the status of the temperature indicator 352. For example, the temperature indicator 352 can be lit to indicate that the pessary 500 can be withdrawn upon receipt of temperature data that indicates that the shape-memory alloy is sufficiently cold that the inner core is in the more compressed second phase 204.

The battery 502 is connected to the processor 504 to provide power for the functioning of the processor 504 and other elements of the pessary 500. In some embodiments, the processor 504 provides any necessary power to the data-transmitting and data-receiving elements in the system. In other embodiments, the battery 502 is connected directly to these other elements including the wireless transmitter 506 or sensors 508, 510, 512.

FIG. 5B illustrates a top view of a ring-shaped vaginal pessary 550 in accordance with various embodiments taught herein. The pessary 550 can include an outer surface 552 that encloses an inner core 554. The pessary 550 can include one or more of a microbial sensor 558, a pH analytic sensor 560, pressure sensors 562, a nerve signal sensor 564, a volume sensor 566, and a drug elution dispenser filament 556. The sensors of the pessary 550 can transmit sensed or measured data to an external computing device in some embodiments. For example, the pessary 550 can include the processor 504 and wireless transmitter/receiver 506 described earlier. In some embodiments, the pessary can include a memory connected to the processor 504 such that the processor can store data in the memory that can be retrieved later once the pessary 550 has been removed from the patient.

In some embodiments, the outer surface 552 can include a silicone casing that is expandable along one or more directions. For example, the outer surface 552 can expand to accommodate lengthening of the inner core 554 including shape-memory materials. In some embodiments, the outer surface 552 can include silver, typically within a carrier medium, embedded into the surface (such as silicone) or silver coating onto the outer surface 552. Silver compounds, such a silver nitrate or silver sulfadiazine (SSD), act as an antimicrobial agent. Thus, a therapeutically effective dose of a carrier fluid or crème containing silver can also be placed in a vial that can be inserted into the sidewall of device and configured to release over a period of time. Another therapeutic agent as described herein can comprise hyaluronic acid that can reduce local edema and maintain a desired moisture level that can be used in combination with other agents. Nanocrystalline silver can also be embedded in a polymer matrix that can provide for gradual release over a selected time period. Biodegradable polymers can also be used to regulate timed release.

The inner core 554 can be formed in a filament-like shape in some embodiments. The shape of the inner core can include a spiral, helix, or spring-like shape that allows an end-to-end length of the inner core 554 to change by a larger amount than if the inner core was a straight wire. The inner core 554 can include shape-memory polymers or shape-memory alloys such as nitinol.

In accordance with various embodiments, a first end and a second end of the pessary overlap or touch. When the ends of the pessary 550 are touching, expansion of the inner core 554 due to temperature changes increases the radius R of the circle while contraction of the inner core 554 due to temperature changes reduces the radius R of the circle. In some embodiments, the reduced radius of the contracted pessary can be in a range from 0.75 cm to 2.5 cm. In some embodiments, the larger radius of the expanded (i.e., installed) pessary can be in a range from 4 to 6 cm. In some embodiments, both ends of the inner core 554 and a space between the two ends of the inner core 554 can be enclosed in the same casement such as if the outer surface 552 of the pessary 550 is formed as a continuous ring. As shown in FIG. 5B, alternative embodiments feature an outer surface that could, topographically speaking, be unfolded into a sheet but that is curled such that the first end of the pessary 550 is tucked behind the second end of the pessary 550. In such embodiments, the first end and the second end of the pessary 550 can be joined or adhered using physical or chemical fasteners in a manner that is either permanent or reversible. In some embodiments, the pessary 550 is shaped as a ring or cylinder having a radius that is not joined along a portion such that opposing sides can expand from a smaller radius to a larger radius upon warming to body temperature.

The pH analytic sensor 560 can collect data about the pH of contacting fluid in the vagina. In some embodiments, the pH analytic sensor 560 is located at a posterior aspect of the device as this location is the most likely location for vaginal fluid to collect. Location at the posterior aspect can optimize data acquisition of pH data in accordance with some embodiments. In some embodiments, the pessary 550 can include at least four pressure sensors 562. The pressure sensors 562 can be distributed evenly around a circumference of the pessary 550 in some embodiments and located at the posterior aspect, an anterior aspect, and both lateral (bilateral) aspects of the pessary 550.

The volume sensor 566 can enable sonographic acquisition of urethra and bladder volume data in some embodiments. For example, the volume sensor 566 can include an ultrasound transducer having one or more transducer elements to produce ultrasound waves that are directed towards the urethra or bladder and an ultrasound receiver array including one or more ultrasound receivers to receive return signals from the urethra or bladder that can be re-constructed into a sonogram. In some embodiments, the volume sensor 566 is located at the anterior aspect of the device such that it is closer to the organs to be imaged.

The microbial sensor 558 can detect the presence of microbes in the vagina such as in vaginal fluid. In some embodiments, the microbial sensor 558 can be located at the posterior aspect of the pessary 550 to optimize acquisition of data where vaginal fluid is most likely to collect.

The drug elution dispenser filament 556 can be inserted into the pessary 550 before insertion of the pessary 550 into the body. In some embodiments, the pessary 550 includes a cavity or channel designed for receiving a filament 556. The outer surface 552 can include pores or openings along at least part of the outer surface 552 to aid in elution of drugs from the filament into the vagina. In some embodiments, the filament 556 can include estradiol or another estrogen-containing drug. The filament 556 can be periodically removed and replaced as the quantity of drug remaining in the filament 556 is reduced by elution. In some embodiments, this is accomplished by removal of the pessary 550 from the body and retrieval of the filament 556 from the channel or cavity using a tool. The new filament 556 can be inserted into the channel and the opening to the channel can be sealed if necessary. The pessary 550 can then be reinserted.

The nerve signal generator 564 can generate an electrical pulse that is directed at sacral spinal nerve S3 in the patient in some embodiments. In some embodiments, the nerve signal generator 564 can be located at the distal posterior aspect of the pessary as this position is closest to the S3 nerve root. The electrical pulse generated by the nerve signal generator 564 can be used to treat bladder or bowel conditions such as overactive bladder. Briefly, the electrical pulse can control afferent signaling pathways to override or block improper nerve signals that indicate to the patient that the bladder or bowel is full. In some embodiments, the nerve signal generator 564 includes an electrode that can deliver a continuous electrical signal over a period of hours, days, weeks, months or years. In some embodiments, the nerve signal generator 564 is responsive to external control from the patient via a remote controller or via control through a computing device, smartphone, or tablet. The nerve signal generator 564 can be communicatively coupled to a control unit 570 mounted on or in the vaginal pessary 550. The control unit 570 can include a processor and a wireless communication device. The external device, such as the handle 320 or a separate computer/smartphone/tablet, is held by the patient or a physician. The patient or physician toggles the electrical pulse on or off using a mechanical or touch control on the external device. The external device communicates with the wireless communication device in the control unit 570 using any of a variety of communication standards including BlueTooth®, Wifi, or rf signaling. The processor that is communicatively coupled to the wireless communication device in the control unit 570 then controls the nerve signal generator 564 to generate the electrical pulse or to terminate generating the electrical pulse according to the signal from the external device. In some embodiments, the nerve pulse generator 564 provides the electrical pulse with a voltage having a range from 0V-10.5V. In some embodiments, the nerve pulse generator 564 provides the electrical pulse with a current in a range of 0.1-15 mA. In some embodiments, the nerve pulse generator 564 provides the electrical pulse at a frequency in a range of 2 to 200 Hz. In some embodiments, the nerve pulse generator 564 provides the electrical pulse with a pulse width in a range from 60-450 microseconds.

Further detail on using electrical signals for incontinence therapy and the types of signal modulation appropriate for such therapy are described in “The science behind programming algorithms for sacral neuromodulation” by Charles Knowles et al., Colorectal Disease, 2021; 23:592-602, in “A prospective, multicenter, international study to explore the effect of three different amplitude settings in female subjects with urinary urge incontinence receiving interstim therapy” by Dean Elterman et al., Neurourology and Urodynamics, 2021; 40:920-928, and in “Posterior tibial nerve stimulation for overactive bladder-techniques and efficacy” by Alka Bhide et al., International Urogynecology Journal (2020) 31:865-870, the entire contents of these three scientific publications being incorporated herein by reference.

In embodiments of vaginal pessaries taught herein, the inner core elements can actuate to change shape (due to changes in temperature) even under loading or when supporting other structures. Examples of actuators including shape-memory alloys that move under loading or while supporting structures can be found in “A Three-Dimensional Constitutive Modeling for Shape Memory Alloys Considering Two-Way Shape Memory Effect and Transformation-Induced Plasticity” by Lei Xu et al., AIAA Scitech 2019 Forum, 6 Jan. 2019, AIAA 2019-1195, and “Thermal study of a shape memory alloy (SMA) spring actuator designed to insure the motion of a barrier structure” by Sonia Degeratu et al., J Therm Anal Calorim; 111:1255-1262; February 2012, the entire contents of each of the above publications being incorporated herein by reference. Systems and methods described herein can employ cooling of shape memory alloys in the inner core of the vaginal pessary to actuate the shape change of the pessary. The shape change behavior under cooling conditions is described in further detail in “Thermoelectric Cooling Of Shape Memory Alloy Actuators: Theoretical Modeling And Experiment” by A. Bhattacharyya et al., Proc. SPIE: Active Materials and Smart Structures, vol. 2427, p. 198-217 (2 Feb. 1995), the entire contents of this application being incorporated herein by reference.

While the inner core materials of vaginal pessaries are described above with respect to nitinol as the shape-memory alloy, it will be understood by the skilled person that other shape-memory materials may also be used in embodiments of the present invention. In particular, shape-memory polymers are contemplated for use as actuators that alternate shape between the first phase 202 and the second phase 204 as described above. Examples of shape-memory polymers are described in “Temperature-memory polymer actuators” by Marc Behl et al., PNAS, vol. 110, no. 31, 12555-12559, Jul. 30, 2013, the entire contents of this publication being incorporated herein by reference.

In different patients there can be different sizes and expansion ratios whereby the difference between the cooled radius of curvature R₁ of the device and the larger radius of curvature R₂ at the body temperature of the patient can be selected to improve therapeutic effectiveness. As it can be desirable to remove and insert the device periodically, the frame can be removed from the biocompatible enclosure and inserted into a new sterile enclosure for re-insertion. The frame can be quickly re-sterilized for this procedure. Alternatively, the entire device can be disposable after each use. An expansion ratio R₂/R₁ defines a metric that characterizes the device that may be suitable for a particular patient. As the normal body temperature T₂ is in the range of 36.4 degrees C. to 37.2 degrees C., the required cooling temperature T₁ can also be in a range depending on the desired expansion ratio. In one example, the temperature T₁ can be 15 degrees C. less than T₂. In this example, at least ten degrees C. of cooling is needed to achieve sufficient contraction of the radius for ease of insertion. In a further example, at least 20 degrees C. or more in temperature change relative to body temperature is needed to obtain sufficient radial contraction. In a further example, at least 25 degrees C. or more in temperature change relative to body temperature is necessary to obtain sufficient radial contraction for insertion. In a further example, at least 30 degrees C. or more in temperature change is needed relative to body temperature to obtain sufficient radial contraction for insertion. In a further example, at least 35 degrees C. or more in temperature change is needed to obtain sufficient radial contraction for insertion. As room temperature is frequently at or about 20-22 degrees C., it can be necessary to cool the device relative to room temperature to achieve sufficient contraction for insertion. In some embodiments, the devices can be stored in a refrigeration device at the required temperature T₁. Alternatively, as described herein with respect to FIGS. 3A and 3B, a delivery applicator can include a thermoelectric cooler that can be used to contact the frame or inner core of the device to provide the necessary cooling to the temperature T₁ that is sufficient to achieve the required radial contraction. In embodiments where the ribs 106 or inner core is formed of nickel-titanium alloys such as nitinol, the material can be annealed as described herein to provide wire or tubular nitinol frames. As is known to one of ordinary skill in the art, wire and tubular structures have known coefficients of thermal expansion with shape memory characteristics over the temperature range from 0 degrees C. to 40 degrees C. In preferred embodiments the smaller radius of the device can be between 1-2 cm and the larger radius of the device can be between 2-5 cm, for example, depending upon the condition of the patient to be treated. The larger diameter is selected so as to facilitate frictional engagement with the surrounding tissue to as to stabilize the position of the device. The smaller diameter is selected to facilitate insertion without risk of abrasion. The outer walls/surfaces will be smooth but can include rounded ridges that assist in limiting longitudinal movement.

A process flow diagram illustrating operative features of preferred embodiments as described herein is shown in FIG. 7. The pessary device can be configured, for example, to enable a user to actuate a nerve stimulation electrode that can be operated automatically upon insertion wherein the processor is programmed to control signal generation for a period of time. The processor can be programmed with the operating parameters for the pessary device, which can also be remotely reset depending upon the therapeutic conditions to be addressed for the individual patient. The one or more sensors can be programmed to periodically transmit and/or record selected sensor data so as to prolong battery life, for example. The sensor system including sensors, processors, and transmitters can be actuated to monitor the effect of therapeutic administration by the pessary device for a selected period of time. Alternatively, the nerve stimulation signal generator can be remotely operated by the user by wireless communication from the handle device used to insert the pessary device, or can be operated by using a cellular phone or tablet device as described herein. The cellular phone or tablet can also display sensor data and can be a web enabled device connectable to a communication network to communication with a physician or other medical provider. As the pessary device can be self-administered and operated by the patient, the communication-enabled devices described herein can be used to remotely monitor patient condition. The one or more sensors can be used to monitor the effects of the delivery of therapeutic agents by the device or that are separately delivered locally or systemically.

The method 700 can include inserting a vaginal pessary 102, 150, 500, 550 including a processor, a memory, and one or more sensors into a vaginal canal of a patient (step 702). The next steps of the method depend upon which sensors or other components are installed on the pessary. Some versions of the method 700 include generating electrical pulses by actuating the nerve signal generator 564 using the processor 504 to deliver nerve washout signal to a sacral spinal nerve root of the patient (step 708). Then, in response to a signal received by the processor 504, 570 from an external device (e.g., handle 320, cellular phone, tablet computer, or other computing device) that communicates with a wireless transmitter/receiver 506 communicatively coupled to the processor 504, discontinue generating electrical pulses (step 710). In other embodiments, the method 700 includes generating sensed data using the one or more sensors 508, 510, 512, 558, 560, 562, 566 (e.g., microbial sensor 558, pH analytic sensor 560, pressure sensor 512, 562, volume sensor 566, sonogram sensor/transmitter) and send sensed data to the processor 504 (step 704). Then, the method 700 includes transmitting sensed data to an external device (e.g., handle 320, cellular phone, tablet computer, or other computing device) using a wireless transmitter/receiver 506 communicatively coupled to the processor 504 (step 706). The method 700 can optionally also include a step of monitoring a condition of the patient based upon the received sensed data in response to delivery of electrical pulses or delivery of therapeutic agent from a reservoir, container, vial, or filament (step 712).

While the present inventive concepts have been described with reference to particular embodiments, those of ordinary skill in the art will appreciate that various substitutions and/or other alterations may be made to the embodiments without departing from the spirit of the present inventive concepts. Accordingly, the foregoing description is meant to be exemplary and does not limit the scope of the present inventive concepts.

A number of examples have been described herein. Nevertheless, it should be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the present inventive concepts.

1. A vaginal pessary device comprising:
a pessary including a thermally actuated shape memory material having a first configuration at a first temperature and a second configuration at a second temperature, the second temperature conforming to a body temperature of a patient, the pessary including an outer surface to contact tissue wherein the first configuration has a first radius and the second configuration has a second radius larger than the first radius.

2. The vaginal pessary device of claim 1, wherein the pessary comprises a hemi-cylindrical shape.

3. The vaginal pessary device of claim 1, wherein the outer surface of the pessary comprises a biocompatible material; and
wherein the shape memory material comprises a metal alloy inner core having a plurality of hemi-cylindrical ribs.

4. The vaginal pessary device of claim 1, wherein an inner core of the pessary comprises a continuous spiral of nitinol helical elements.

5. The vaginal pessary device of claim 1, further comprising an applicator device having a cooling element of a thermoelectric cooler.

6. The vaginal pessary of claim 3, wherein the biocompatible material comprises silicone.

7. The vaginal pessary device of claim 1, wherein the pessary comprises an inner core having a set of nitinol core elements.

8. The vaginal pessary device of claim 7, wherein the set of nitinol core elements are treated at a temperature of 805° C. at a treatment duration of 5 minutes or wherein the set of nitinol core elements are treated at a temperature of 550° C. at a treatment duration of 5 minutes, or wherein the set of nitinol core elements are treated at a temperature of 400° C. at a treatment duration of 5 minutes, or wherein the set of nitinol core elements are treated at a temperature of 805° C. at a treatment duration of 30 minutes.

9-11. (canceled)

12. The vaginal pessary device of claim 1 further comprising at least one of a temperature sensor and a pressure sensor.

13. (canceled)

14. The vaginal pessary device of claim 1 further comprising a drug dispenser.

15. The vaginal pessary device of claim 14 wherein the drug dispenser comprises a container that can be inserted into a reservoir of the pessary, the container having a cavity to contain a drug to be dispensed into the body.

16. The vaginal pessary device of claim 14 wherein the drug comprises a fluid.

17. The vaginal pessary device of claim 15 wherein the drug comprises estradiol.

18. The vaginal pessary device of claim 1 further comprising a sensor, a battery, a processor, a memory and a wireless transmitter.

19. (canceled)

20. The vaginal pessary device of claim 1 wherein the device is shaped and sized for insertion into the vaginal canal and has lateral sides for contacting walls of the vaginal canal.

21. (canceled)

22. The vaginal pessary device of claim 1 wherein the first temperature is at least 25 degrees C. cooler than the body temperature or wherein the first temperature is at least 30 degrees C. cooler than the body temperature or wherein the first temperature is at least 35 degrees C. cooler than the body temperature.

23. (canceled)

24. (canceled)

25. The vaginal pessary device of claim 1 wherein the second temperature is in a range from 36.4 and 37.2 degrees C.

26. The vaginal pessary device of claim 18, wherein the sensor measures a parameter in response to dispensing of a therapeutic agent.

27. The vaginal pessary device of claim 1 wherein the device comprises a ring or a cylinder having a radius that is not joined along a portion such that opposing sides can expand from a smaller radius to a larger radius upon warming to the body temperature.

28. A method of inserting a vaginal pessary comprising:
cooling a shape memory material of the vaginal pessary to reduce a temperature of one or more core elements of the vaginal pessary; and
inserting the cooled vaginal pessary into a position upon a surface of a vaginal wall of a body such that the temperature of the vaginal pessary increases to a body temperature wherein the core elements change configuration in response to the increase in temperature and thereby immobilize the vaginal pessary relative to the vaginal wall.

29. The method of claim 28, wherein the inserting step further comprises coupling a cooling element comprising a thermoelectric cooler to the one or more core elements.

30. The method of claim 28, wherein the one or more core elements of the vaginal pessary comprises a set of nitinol core elements.

31. The method of claim 28, wherein the set of nitinol core elements comprise a set of hemi-cylindrical ribs or wherein the set of nitinol core elements comprise a continuous spiral of helical elements.

32. (canceled)

33. The method of claim 28, further comprising:
causing a notification element on an applicator to display a notification responsive to the determining that the temperature of the one or more core elements of the vaginal pessary have reached the threshold temperature.

34. The method of claim 33, wherein the applicator comprises a clamp configured to compress the vaginal pessary and further comprising causing the cooling element of the applicator to reduce the temperature of the one or more core elements of the vaginal pessary and thereby causing the one or more core elements to transition from a first phase to a second phase based on the temperature of the one or more core elements.

35. (canceled)

36. The method of claim 28, wherein the vaginal pessary comprises a hemi-cylindrical shape.

37. The method of claim 28 further comprising determining a temperature of the one or more core elements of the vaginal pessary.

38. The method of claim 28 further comprising:
sensing the temperature and determining if the sensed temperature is above a threshold temperature; and
removing an applicator.

39. The method of claim 28 further comprising dispensing a drug from the vaginal pessary and sensing a condition of the patient in response to delivery.

40. A vaginal pessary system comprising:
a vaginal pessary, including:
an outer surface comprising a biocompatible material, and
an inner core comprising a shape-memory alloy; and
an applicator device including a cooling element and a clamp.

41. The vaginal pessary system of claim 40, wherein the vaginal pessary has a hemi-cylindrical shape.

42. The vaginal pessary system of claim 40, wherein the inner core of the vaginal pessary comprises a set of hemi-cylindrical nitinol ribs or wherein the inner core of the vaginal pessary comprises a continuous spiral of nitinol helical elements.

43. (canceled)

44. The vaginal pessary system of claim 40, wherein the cooling element of the applicator device comprises a thermoelectric cooler.

45. The vaginal pessary system of claim 40, wherein the biocompatible material comprises silicone.

46. The vaginal pessary system of claim 40, wherein the inner core of the vaginal pessary comprises a set of nitinol core elements, wherein the set of nitinol core elements are treated at a temperature of 805° C. at a treatment duration of 5 minutes or wherein the set of nitinol core elements are treated at a temperature of 550° C. at a treatment duration of 5 minutes or wherein the set of nitinol core elements are treated at a temperature of 400° C. at a treatment duration of 5 minutes or, wherein the set of nitinol core elements are treated at a temperature of 805° C. at a treatment duration of 30 minutes

47-50. (canceled)

51. The method of claim 28 further comprising:
determining that the temperature of the one or more core elements of the vaginal pessary have reached a threshold temperature; and
removing the applicator.

52-59. (canceled)

60. A vaginal pessary device comprising:
a shape memory material having a first configuration at a first temperature and a second configuration at a second temperature, the second temperature conforming to a body temperature of a patient, the pessary device including an outer surface to contact tissue wherein the first configuration has a first radius and the second configuration has a second radius larger than the first radius; and
a sensor to detect a condition of the pessary device.

61. The vaginal pessary of claim 60, wherein the vaginal pessary comprises a hemi-cylindrical shape.

62. The vaginal pessary of claim 60, wherein the outer surface of the pessary device comprises a biocompatible material; and
wherein the shape memory material comprises a metal alloy inner core having a plurality of hemi-cylindrical ribs or wherein an inner core of the vaginal pessary comprises a continuous spiral of nitinol helical elements.

63-66. (canceled)

67. The vaginal pessary of claim 60 further comprising a therapeutic agent for delivery to the patient.

68. The vaginal pessary of claim 67 wherein the sensor detects a condition of the patient in response to delivery of the therapeutic agent.

69. The vaginal pessary of claim 67 further comprising a processor configured to set a value for an operating parameter of a sensor processing circuit on the device that is configured to remotely controlling an operating parameter of the sensor using a processor and a wireless receiver with a cellular phone or tablet.

70. (canceled)
